(19) United States
(12) Patent Application Publication
(10) Pub. No.: US 2024/0034981 A1
(43) Pub. Date: Feb. 1, 2024

(54) Devices and Methods for Evaluating the Viability of Embryos

(71) Applicant: The Regents of the University of California, Oakland, CA (US)

(72) Inventors: Paolo Rinaudo, San Francisco, CA (US); Seok Hee Lee, San Francisco, CA (US)

(21) Appl. No.: 18/027,292

(22) PCT Filed: Oct. 21, 2021
(86) PCT No.: PCT/US2021/056046

§ 371 Date: Mar. 20, 2023

Related U.S. Application Data
(60) Provisional application No. 63/126,108, filed on Dec. 16, 2020.
(60) Provisional application No. 63/104,228, filed on Oct. 22, 2020.

(51) Int. Cl. C12M 1/34; C12N 5/073

(52) U.S. Cl. CPC C12M 41/26; C12N 5/0604; C12M 41/38

(57) ABSTRACT

Devices, systems, and methods for evaluating the viability of embryos are disclosed. In particular, devices, systems, and methods for measuring internal and external pH of an embryo, electrolytes, and oxidative stress markers for evaluating developmental potential of an embryo are provided. The methods disclosed herein should improve in vitro fertilization (IVF) outcomes by reducing inadvertent transfer of non-viable embryos.

### CROSS-REFERENCE TO RELATED APPLICATIONS

This application claims the benefit of U.S. Provisional Patent Application No. 63/104,228, filed Oct. 22, 2020, and U.S. Provisional Patent Application No. 63/126,108, filed Dec. 16, 2020, which applications are incorporated herein by reference in their entirety.

### STATEMENT REGARDING FEDERALLY SPONSORED RESEARCH OR DEVELOPMENT

This invention was made with government support under grant no. R01 HD092267 awarded by The National Institutes of Health. The government has certain rights in the invention.

### BACKGROUND OF THE INVENTION

Twelve percent of American women 15 to 55—7.3 million—have used some sort of fertility service; the use of assisted reproductive technologies has doubled in the past decade. In 2015, these procedures resulted in nearly 73,000 babies—1.6 percent of all U.S. births. The rate is even higher in some countries, including Japan (5 percent) and Denmark (10 percent). IVF, i.e. the fertilization of egg and sperm in vitro with the resulting embryo being transferred to the patient, has resulted in the birth of 8 million children worldwide (Adamson, et al. 2018). Unfortunately, despite advances made in IVF technology, more than 85% of embryos produced by IVF are not viable and multiple cycles of IVF are often needed to conceive, and therefore, multiple embryos are transferred in a single cycle. This practice results in a major increase in the number of multiple births which significantly compromise maternal and newborn health. Consequently, the cost of medical care associated with multiple pregnancies greatly exceeds the cost of the IVF treatment itself. In addition, embryos generated outside of the body are exposed to non-physiologic conditions that profoundly affect their future development. In fact, according to the Developmental Origins of Health and Disease (DOHaD) hypothesis, during critical periods in development, organisms exhibit an enhanced plasticity that enables them to fine-tune patterns of gene expression in accordance with environmental cues. Such changes often confer immediate survival advantages; however, they may also induce inappropriate adaptive changes that conflict with postnatal environments and consequently impair adult metabolic health (Bateson, et al. 2004).

IVF done with mouse embryos is associated with lags in developmental timing, decreased rate of blastocyst formation, and fewer cells (Feuer, et al. 2016, Rinaudo and Schultz 2004). Further, abnormal mouse placental growth with altered fetal placenta ratio (Delle Piane, et al. 2010) and impaired amino acid transport to the fetus (Bloise, et al. 2012) is also problematical. Postnatally, IVF offspring develop glucose intolerance in adulthood and exhibit evidence of β-cell dysfunction (Donjacour, et al. 2014, Feuer, et al. 2014). The more abnormal the culture conditions used, the more severe the uterine and postnatal phenotype.

While human studies are limited, there is mounting evidence suggesting an increased incidence of health complications, including hypertension and glucose intolerance in IVF offspring (Ceelen, et al. 2008, Meister, et al. 2018, Rimoldi, et al. 2014).

There is therefore a great need to develop technologies that identify the healthiest embryo to transfer, with higher chance of implantation and in resulting in the birth of a healthy child.

### SUMMARY OF THE INVENTION

Devices, systems, and methods for evaluating the viability of embryos are disclosed. In particular, devices, systems, and methods for measuring internal and external pH of an embryo, electrolytes, and oxidative stress markers for evaluating developmental potential of an embryo are provided. The methods disclosed herein should improve in vitro fertilization (IVF) outcomes by reducing inadvertent transfer of non-viable embryos. Additionally, the subject methods may substantially increase the number of successful first-time IVF cycles without risking multiple births.

In one aspect, a system for measuring pH of an embryo in in vitro culture is provided, the system comprising: a) a device comprising a reservoir for holding an embryo; b) a pH sensor, wherein the pH sensor is located at the bottom of the reservoir; and c) a sensor reader that display information from the pH sensor, wherein the sensor reader is connected to the pH sensor (e.g., by a wire or wireless connection).

In certain embodiments, the system further comprises one or more sensors for measuring one or more electrolytes, including, without limitation lactate, pyruvate, or a combination thereof.

In certain embodiments, the system further comprises an oxygen sensor.

In certain embodiments, the system further comprises a temperature sensor.

In certain embodiments, the system further comprises an incubator, a microscope, culture media, or a combination thereof.

In another aspect, a kit comprising a system for measuring pH of an embryo in in vitro culture, as described herein, is provided.

In certain embodiments, the kit further comprises instructions for using the culture dish for measuring external pH while culturing an embryo to evaluate the viability of an embryo.

In certain embodiments, the kit further comprises at least one reagent for measuring levels of an oxidative stress marker.

In certain embodiments, the kit further comprises further comprises at least one reagent for measuring levels of lactate or pyruvate.

In certain embodiments, the kit further comprises media suitable for culturing an embryo.

In another aspect, a method of measuring pH during in vitro culture of an embryo and implanting the embryo into a female recipient, if the embryo is not determined to have poor developmental potential based on the measured pH, is provided, the method comprising: a) culturing an embryo in vitro in a culture media under conditions suitable for embryo development in the reservoir of the system described herein; b) measuring pH of the culture media in the reservoir during development of the embryo at one or more time points using the pH sensor of the system described herein, wherein a lower pH of the culture media compared to a reference value indicates that the embryo has poor developmental potential; and c) implanting the embryo into a female recipient if the embryo is not determined to have poor developmental potential based on the pH. In certain embodiments, the method further comprises measuring one or more markers of oxidative stress in a cell of the embryo, wherein lower pH of the culture media in combination with higher levels of the one or more markers of oxidative stress compared to reference value ranges for a healthy embryo indicate that the embryo has poor developmental potential. In certain embodiments, the method further comprises measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with lower pH of the culture media and higher levels of the one or more markers of oxidative stress compared to reference value ranges for a healthy embryo indicate that the embryo has poor developmental potential. In certain embodiments, the embryo is human.

In another aspect, a method for evaluating the developmental potential of an embryo is provided, the method comprising measuring internal pH of the embryo during development, wherein a higher internal pH of the embryo compared to a reference level indicates that the embryo has poor developmental potential. In certain embodiments, the method further comprises measuring one or more markers of oxidative stress in a cell of the embryo, wherein higher internal pH in the embryo in combination with higher levels of the one or more markers of oxidative stress compared to reference value ranges for a healthy embryo indicate that the embryo has poor developmental potential. In certain embodiments, the method further comprises measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with higher internal pH in the embryo and higher levels of the one or more markers of oxidative stress compared to reference value ranges for a healthy embryo indicate that the embryo has poor developmental potential.

The disclosed methods should enable more successful initial IVF cycles (i.e., higher pregnancy rates), be more cost efficient, less risky, and a more attractive option than the current reliance on multiple IVF cycles.

The subject methods may be used alone or in combination with any other method of embryo assessment, including, but not limited to measuring one or more cellular parameters, measuring gene expression levels of one or more genes in the embryo, genotyping the embryo, detecting aneuploidy, quantitating blastomere fragmentation, detecting blastomere asymmetry, or evaluating the morphology of the embryo in culture (e.g., at days 2, 3, 4, 5, or 6), or any combination thereof.

### BRIEF DESCRIPTION OF THE DRAWINGS

FIGS. 1A-1B. Internal pH of individual mouse blastocysts was measured using the die SNURF1; external pH was measured with a highly sensitive pH probe in the culture drop were 20 embryos developed from the zygote stage to the blastocyst stage for 4.5 days. Embryos that develop following in vitro fertilization (IVF) and cultured in more stressful conditions (20% Oxygen>5% Oxygen) display higher internal pH (FIG. 1A) and lower external pH in the secreted media (FIG. 1B). Because control embryos (=FB) are flushed out of the uterus and not cultured, there is no pHe value. Error bars represents SD. Different superscript indicate p<0.05.

FIGS. 2A-2C. Analysis of oxidative damage using immunofluorescence in mouse blastocysts generated in vivo (flushed blastocysts=FB=control) or by IVF with different oxygen concentrations (5% O₂ or physiologic and 20% O₂ or stressful). Results show that there is increased (FIG. 2A) DNA (8-hydroxy-deoxy-guanosine 8-OHdG), (FIG. 2B) lipid (F2α isoprostane) and (FIG. 2C) protein (carbonyl group) oxidative damage in IVF embryos. Further, more stressful culture conditions (20% Oxygen>5%) results in more significant oxidative damage. In separate published work we have shown that embryos cultured in IVF 20% have an abnormal placenta/fetal growth, and abnormal glucose levels in adult life. Error bars represent SD. Different superscript indicate p<0.05.

FIGS. 3A-3B. Schematic showing design of system with a pH sensor for measuring pH of media surrounding an embryo in in vitro culture. FIG. 3A shows examples of current dishes used to culture mammalian embryos. FIG. 3B shows a schematic of a modified dish for culturing mammalian embryos that includes one or more sensors on the bottom of the reservoir for measuring pH, specific electrolytes (e.g., lactate, pyruvate), or markers of oxidative stress or damage. In the example shown, the dish has sensors added to the bottom, with wires that connect to a pH reader outside of the incubator (not drawn to scale).

FIGS. 4A-4D. Lactate and pyruvate levels in embryos and culture medium of embryos. Intracellular lactate levels increased with increasing suboptimal culture conditions (FIG. 4A). This signifies that the healthiest embryo can be identified based on intracellular lactate levels. A healthy IVF-generated embryo has a lactate level closer to an in vivo (flashed blastocyst (FB)=control) embryo. Extracellular lactate levels decreased with increasing suboptimal culture conditions (FIG. 4C). This means that extracellular level of lactate can be used to identify the healthiest embryo. Healthiest embryos have lower lactate levels in the culture medium. Intracellular pyruvate levels decreased with increasing suboptimal culture conditions (FIG. 4B). This signifies that healthiest embryo can be identified based on intracellular pyruvate levels. A healthy IVF-generated embryo has a pyruvate level closer to an in vivo (flashed blastocyst (FB)=control) embryo. Extracellular pyruvate levels did not change significantly (ND=not different statistically) with increasing suboptimal culture conditions (FIG. 4D). This means that extracellular levels of pyruvate cannot be used to identify the healthiest embryo.

### DETAILED DESCRIPTION OF THE INVENTION

Devices, methods, and systems for evaluating the viability of embryos are disclosed. In particular, devices, methods, and systems for measuring internal or external pH of an embryo, electrolytes, and oxidative stress markers for evaluating developmental potential of an embryo are provided.

Before the present devices, methods, and systems are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “an embryo” includes a plurality of such embryos and reference to “the marker” includes reference to one or more markers and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “developmental potential” and “developmental competence” are used herein to refer to the ability or capacity of a healthy embryo or pluripotent cell to grow or develop.

The term “embryo” is used herein to refer both to the zygote that is formed when two haploid gametic cells, e.g. an unfertilized secondary oocyte and a sperm cell, unite to form a diploid totipotent cell, e.g. a fertilized ovum, and to the embryo that results from the immediately subsequent cell divisions, i.e. embryonic cleavage, up through the morula, i.e. 16-cell stage and the blastocyst stage (with differentiated trophoectoderm and inner cell mass).

The terms “subject,” “individual,” and “patient” are used interchangeably herein to refer to a mammal to whom an embryo produced by in vitro fertilization is transferred. In an embodiment, the mammal is a human, such as a female human. The terms “subject,” “individual,” and “patient” also encompass individuals in need of treatment for clinical infertility, including those who have undergone or are candidates for an in vitro fertilization cycle.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

The term “oxidative stress marker” as used herein refers to a compound, molecule, or radical, such as a peroxide, superoxide, hydroxyl radical, singlet oxygen, alpha-oxygen, hydrogen peroxide, nitric oxide, an oxidized or nitrated protein, DNA, RNA, or lipid; or a reaction product thereof that is present at higher concentrations, levels or frequencies in one sample compared to another, such as a sample from an embryo having oxidative stress compared to one or more samples from healthy control embryos (i.e., embryos not having oxidative stress). Oxidative stress markers include, but are not limited to, protein oxidative stress markers such as, proteins with carbonyl oxidation (e.g., including carbonyl derivatives of proline, lysine, arginine and threonine residues) and protein radicals generated from electron transfer from reactive oxygen or reactive nitrogen species; DNA/RNA oxidative stress markers such as 8-hydroxyguanosine (8-OHG), 8-hydroxydeoxyguanosine (8-OHdG), 8-nitroguanine, abasic (AP) sites, and etheno-DNA adducts such as ethenoadenosine, ethenocytidine, ethenoguanine, ethenodeoxyadenosine, ethenodeoxycytidine, and ethenodeoxyguanosine; and lipid oxidative stress markers such as lipid peroxides, 8-iso-prostaglandin F2alpha (8-isoprostane), 4-hydroxynonenal (HNE), and malondialdehyde (MDA).

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the marker. These values or ranges can be obtained from a single patient or from a group of patients.

The term “assaying” is used herein to include the physical steps of manipulating a biological sample (e.g., sample from embryo comprising oxidative stress marker, pyruvate, or lactate) to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from an embryo. Accordingly, a biological sample can be isolated from an embryo (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample. Methods and protocols for isolating biological samples will be known to one of ordinary skill in the art and any convenient method may be used to isolate a biological sample. In some embodiments, a biological sample comprising one or more cells is obtained from an embryo for preimplantation testing (e.g., genetic testing, measuring cellular levels of oxidative stress markers, pyruvate levels, lactate levels, gene expression levels, cell parameters, etc.).

It will be understood by one of ordinary skill in the art that in some cases, it is convenient to wait until multiple samples (e.g., from multiple embryos) have been obtained prior to assaying the samples. Accordingly, in some cases an isolated biological sample is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a sample from one embryo is assayed prior to obtaining a sample from another embryo. In some cases, sample from multiple embryos are assayed in parallel. In some cases, multiple different samples from embryos generated by IVF of oocytes from the same female donor subject or different female donor subjects are assayed in parallel. In some cases, biological samples are processed immediately or as soon as possible after they are obtained.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the level of a marker is less than or “greater than or equal to” a particular threshold, (the threshold can be pre-determined or can be determined by assaying a control sample). On the other hand, “assaying to determine the level of a marker” can mean determining a quantitative value (using any convenient metric) that represents the level of the marker (i.e., amount of an oxidative stress marker, pyruvate, or lactate, expression level, e.g., the amount of protein and/or RNA, e.g., mRNA) of a particular marker. The level of a marker can be expressed in arbitrary units associated with a particular assay (e.g., fluorescence units, e.g., mean fluorescence intensity (MFI)), or can be expressed as an absolute value with defined units (e.g., number of reactive oxygen species, number of oxidized protein, DNA, RNA, or lipid molecules, number of mRNA transcripts, number of protein molecules, concentration of protein, etc.). Additionally, the level of a marker can be compared to the level of one or more additional markers to derive a normalized value that represents a normalized level. The specific metric (or units) chosen is not crucial as long as the same units are used (or conversion to the same units is performed) when comparing multiple embryos.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what embryo or type of biological sample(s) was used and/or how it was used, the technique used in measurements (e.g., for measuring pH, electrolytes, or oxidative stress), the results of the measurements, the assessment as to whether the embryo is determined to be healthy/viable or unhealthy/stressed, a recommendation as to whether the embryo should be transferred to a female subject, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Devices and Systems for Evaluating the Viability of an Embryo

Devices and systems for evaluating the viability of embryos are disclosed. Embryos, generated by in vitro fertilization (IVF), which are cultured under suboptimal culture conditions (e.g., stressful or non-physiological conditions, >5% oxygen), display a higher internal pH than normal healthy embryos that develop under normal physiological conditions. While not being bound by a particular theory, the external pH of the media surrounding stressed/unhealthy embryos decreases during in vitro culture because stressed/unhealthy embryos, having a higher internal pH than normal/healthy embryos, secrete more H⁺ ions into the surrounding media resulting in a lower external pH. Accordingly, the external pH of the media surrounding an embryo in a culture dish can be measured non-invasively to provide an indirect measure of the internal pH and viability of an embryo.

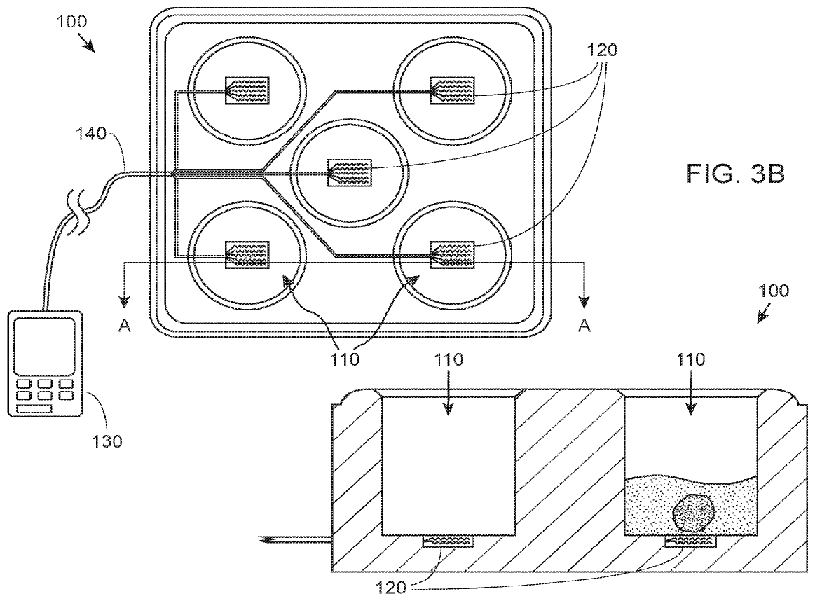

Custom culture dishes are provided for monitoring external pH in media during culture of an embryo. As shown in FIG. 3B, an exemplary custom culture dish 100 comprises one or more wells 110 containing sensors 120 for monitoring pH of media surrounding an embryo (i.e., external pH). In such custom culture dishes, each well 110 holds a single embryo, and the bottom surface of each well has a pH sensor 120 such that the external pH of an individual embryo can be measured. The pH sensor 120 may be connected to an external sensor reader 130 through a wire or wireless connection 140. In some embodiments, the external pH of a group of embryos is measured in a single dish, wherein each embryo is cultured in a separate well having its own pH sensor to allow the external pH of each embryo to be measured separately. In some embodiments, the culture dish further comprises one or more additional sensors for monitoring parameters other than pH, for example, including, without limitation, sensors for measuring temperature, oxygen levels, and/or the concentration of electrolytes (e.g., lactate, pyruvate) in media surrounding an embryo in a well.

In some embodiments, the custom culture dish comprises at least 2 wells, at least 4 wells, at least 6 wells, at least 8 wells, at least 10 wells, at least 15 wells, at least 20 wells, at least 30 wells, at least 40 wells, at least 50 wells, at least 60 wells, at least 80 wells, at least 100 wells, or more. In some embodiments, the custom culture dish comprises between 1 and 25 wells, including any number of wells within this range, such as 1, 2, 3, 4, 5, 6, 7, 8, 9, 10, 11, 12, 13, 14, 15, 16, 17, 18, 19, 20, 21, 22, 23, 24, or 25 wells per dish.

In some embodiments, at least 2 embryos, at least 4 embryos, at least 6 embryos, at least 8 embryos, at least 10 embryos, at least 15 embryos, at least 20 embryos, at least 30 embryos, at least 40 embryos, at least 50 embryos, at least 60 embryos, at least 80 embryos, at least 100 embryos, or more are cultured per dish. In some embodiments, between 1 and 25 embryos are cultured per dish, including any number of embryos within this range, such as 1, 2, 3, 4, 5, 6, 7, 8, 9, 10, 11, 12, 13, 14, 15, 16, 17, 18, 19, 20, 21, 22, 23, 24, or 25 embryos per dish.

The embryos monitored in the culture system may be derived from any organism, e.g. any mammalian species, e.g. human, primate, equine, bovine, porcine, canine, feline, etc. In some embodiments, the embryos are derived from a human. The embryos may be previously frozen, e.g. embryos cryopreserved at the 1-cell stage and then thawed, or frozen and thawed oocytes. Alternatively, the embryos may be freshly prepared, e.g., embryos that are freshly prepared from oocytes by in vitro fertilization or intracytoplasmic sperm injection techniques; oocytes that are freshly harvested and/or freshly matured through in vitro maturation techniques or that are derived from stem cells differentiated in vitro into germ cells and matured into oocytes; and the like.

The embryos may be cultured under any convenient conditions known in the art to promote survival, growth, and/or development of the embryo to be assessed, e.g., for embryos, under conditions such as those used in the art of in vitro fertilization or intracytoplasmic sperm injection; see, e.g., U.S. Pat. Nos. 6,610,543, 6,130,086, 5,837,543, the disclosures of which are incorporated herein by reference; for oocytes, under conditions such as those used in the art to promote oocyte maturation; see, e.g., U.S. Pat. Nos. 5,882,928 and 6,281,013, the disclosures of which are incorporated herein by reference; for stem cells under conditions such as those used in the art to promote proliferation, see, e.g. U.S. Pat. Nos. 6,777,233, 7,037,892, 7,029,913, 5,843,780, and 6,200,806, US Application No. 2009/0047263; US Application No. 2009/0068742, the disclosures of which are incorporated herein by reference. Often, the embryos cells are cultured in a commercially available medium such as KnockOut DMEM, DMEM-F12, or Iscoves Modified Dulbecco's Medium that has been supplemented with serum or serum substitute, amino acids, and growth factors tailored to the needs of the particular embryo being assessed. During culture in the custom culture dish, one or more developing embryos may be cultured in the same or different culture media.

In some embodiments, the pH sensor comprises an electrode that measures pH and detects changes in pH in the culture media during development of the embryo. In some embodiments, the pH sensor comprises a combination electrode comprising a measuring electrode and a reference electrode.

In other embodiments, the pH sensor comprises a solid support functionalized with a pH indicator such as a pH-sensitive dye. The solid support may include, without limitation, a microbead, a microparticle, a culture plate well, a glass plate, and the like. Exemplary pH indicators, include, without limitation, bromothymol blue, thymol blue, bromophenol blue, bromocresol purple, azolitmin (litmus), phenol red, neutral red, phenolphthalein, naphtholphthalein, thymolphthalein, and cresol red. For a description of pH-sensing gel microbeads functionalized with the pH indicator bromothymol blue, see, e.g., Maruyama et al. (2008) Lab Chip 8(2):346-351; herein incorporated by reference in its entirety.

The pH of an embryo may be monitored continuously or intermittently during development in culture. In some embodiments, the external pH of an embryo is measured at one or more timepoints during culture of the embryo up until the time of transfer to a female subject. For example, external pH of an embryo may be measured at one or more time points from the zygote stage to the blastocyst stage, or longer up until the time of transfer to a female subject to determine if the embryo is stressed or unhealthy. Culture conditions may be adjusted if the external pH of an embryo indicates that the culture conditions are stressful to the embryo based on the measured external pH. In cases where multiple embryos are cultured in the same dish, the external pH can be measured for all embryos simultaneously or individual embryos sequentially.

In some embodiments, the external pH of an embryo is measured during culture of an embryo continuously or intermittently for 1 to 7 days or longer (i.e., up to the day of transfer to a female subject), including any number of days within this range such as 1 day, 2, days, 3 days, 4 days, 5 days, 6 days, or 7 days, or longer.

In some embodiments, the culture dish further comprises one or more sensors for measuring one or more electrolytes. In some embodiments, the culture dish further comprises one or more biosensors for measuring pyruvate, lactate, or a combination thereof.

### Oxidative Stress Markers

Oxidative stress markers that can be used in the practice of the subject methods include, without limitation, compounds, molecules, or radicals, such as a peroxide, superoxide, hydroxyl radical, singlet oxygen, alpha-oxygen, hydrogen peroxide, nitric oxide, oxidized or nitrated proteins, DNA, RNA, or lipids; or reaction products thereof that are present at higher concentrations, levels or frequencies in one sample compared to another, such as a sample from an embryo having oxidative stress compared to one or more samples from healthy control embryos (i.e., embryos not having oxidative stress). Oxidative stress markers include, but are not limited to, protein oxidative stress markers such as, proteins with carbonyl oxidation (e.g., including carbonyl derivatives of proline, lysine, arginine and threonine residues) and protein radicals generated from electron transfer from reactive oxygen species or reactive nitrogen species; DNA/RNA oxidative stress markers such as 8-hydroxyguanosine (8-OHG), 8-hydroxydeoxyguanosine (8-OHdG), 8-nitroguanine, abasic (AP) sites, and etheno-DNA adducts such as ethenoadenosine, ethenocytidine, ethenoguanine, ethenodeoxyadenosine, ethenodeoxycytidine, and ethenodeoxyguanosine; and lipid oxidative stress markers such as lipid peroxides, 8-iso-prostaglandin F2alpha (8-isoprostane), 4-hydroxynonenal (HNE), and malondialdehyde (MDA).

Increased levels of these markers are associated with oxidative stress. Accordingly, monitoring the levels of these markers is useful for identifying embryos having oxidative stress. One or more oxidative stress markers may be measured in a cell obtained from the embryo prior to implantation in a female subject. In some embodiments, one or more cells are obtained simultaneously from the embryo for preimplantation genetic testing (PGT) and measurement of levels of oxidative stress markers.

For detecting oxidative stress in an embryo, for example, when analyzing the levels of oxidative stress markers in cell sample from the embryo, the reference value ranges used for comparison can represent the levels of one or more markers in a cell sample from one or more embryos without oxidative stress (i.e., normal or healthy control embryo), wherein detection of increased levels of one or more markers of oxidative stress selected from the group consisting of proteins with carbonyl oxidation, protein radicals, 8-hydroxyguanosine (8-OHG), 8-hydroxydeoxyguanosine (8-OHdG), 8-nitroguanine, abasic (AP) sites, and etheno-DNA adducts such as ethenoadenosine, ethenocytidine, ethenoguanine, ethenodeoxyadenosine, ethenodeoxycytidine, and ethenodeoxyguanosine; and lipid oxidative stress markers such as lipid peroxides, 8-iso-prostaglandin F2alpha (8-isoprostane), 4-hydroxynonenal (HNE), and malondialdehyde (MDA) in the cell sample from the embryo compared to reference values ranges for the levels of the oxidative stress markers in a control sample indicate the embryo has oxidative stress. Alternatively, the reference values can represent the levels of one or more markers in a cell sample from one or more embryos with oxidative stress, wherein similarity to the reference value ranges indicates the severity of oxidative stress.

In some cases, combinations of oxidative stress markers are used in the subject methods. In some such cases, the levels of all measured markers of oxidative stress must change (as described above) in order for the determination to be made that the individual embryo has oxidative stress. In some embodiments, only some markers selected from proteins with carbonyl oxidation, protein radicals, 8-hydroxyguanosine (8-OHG), 8-hydroxydeoxyguanosine (8-OHdG), 8-nitroguanine, abasic (AP) sites, and etheno-DNA adducts such as ethenoadenosine, ethenocytidine, ethenoguanine, ethenodeoxyadenosine, ethenodeoxycytidine, and ethenodeoxyguanosine; and lipid oxidative stress markers such as lipid peroxides, 8-iso-prostaglandin F2alpha (8-isoprostane), 4-hydroxynonenal (HNE), and malondialdehyde (MDA) are used in the methods described herein. For example, a single marker, two types of markers, three types of markers, four types of markers, or five types of markers can be used in any combination. In other embodiments, all the markers of oxidative stress are used.

### Pyruvate and Lactate as Markers of Embryo Stress

Under suboptimal culture conditions, stressed embryos exhibit higher intracellular levels of lactate, lower extracellular levels of lactate, and lower intracellular levels of pyruvate. Accordingly, monitoring the levels of lactate and pyruvate is useful for identifying stressed unhealthy embryos. Intracellular levels of lactate, pyruvate, or a combination thereof may be measured in a cell sample obtained from the embryo prior to implantation in a female subject. In some embodiments, one or more cells are obtained simultaneously from the embryo for preimplantation genetic testing (PGT) and measurement of intracellular levels of pyruvate and lactate. Extracellular levels of lactate may be measured in culture media surrounding an embryo, for example, with a lactate biosensor located in the culture reservoir.

### Detecting and Measuring Markers

It is understood that the stress markers (e.g., oxidative stress markers, pyruvate, and lactate) in a sample can be measured by any suitable method known in the art. For antibody-based methods of detecting and quantitating markers, any convenient antibody can be used that specifically binds to the intended marker. The terms “specifically binds” or “specific binding” as used herein refer to preferential binding to a molecule relative to other molecules or moieties in a solution or reaction mixture (e.g., an antibody specifically binds to a particular marker or epitope relative to other available molecules or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower K_{d}.

While a variety of different manners of assaying levels of markers are known in the art, one representative and convenient type of protocol for assaying levels of markers is the enzyme-linked immunosorbent assay (ELISA). In ELISA and ELISA-based assays, one or more antibodies specific for the oxidative stress markers of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°−27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more markers in a sample may be employed and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), immunoblots, and immunoprecipitation assays; mass spectrometry (MS), tandem mass spectrometry (MS/MS), and tandem liquid chromatography mass spectrometry (LC-MS/MS). As with ELISAs, antibodies (e.g., monoclonal antibodies) that specifically bind to the markers of interest are used in such methods.

In some cases, non-antibody-based methods may be used for measuring a marker of embryo stress. For example, apurinic or apyrimidinic (AP or abasic) sites can be detected with an aldehyde reactive probe that reacts with an aldehyde group on the open ring of the AP site. The AP site is subsequently labeled, for example, with biotin, which can be detected with a streptavidin-enzyme conjugate. Pyruvate can be measured, for example, with an enzymatic assay using lactate dehydrogenase or pyruvate oxidase, or with a pyruvate biosensor. Lactate can be measured, for example, with an enzymatic assay using lactate dehydrogenase or a lactate biosensor.

The resultant data provides information regarding the amount of each marker of embryo stress that is measured. The information may be in terms of whether or not the marker is present and at what level, wherein the data may be both qualitative and quantitative.

### Ranking Embryos for Transfer

The methods described herein may be used to guide clinical decisions, such as whether or not to transfer an in vitro fertilized embryo to a female subject and allow physicians to avoid transfer of unhealthy or non-viable embryos likely to result in miscarriage. Assessments of embryo viability made by the subject methods may also find use in ranking embryos in a group of embryos to allow selection of the healthiest embryos for transfer to a female subject. For example, in some instances, multiple embryos may be capable of developing into blastocysts, i.e. will have favorable developmental potential. However, some embryos will be healthier and less stressed than others, as indicated by having an external pH in a normal reference range. If one or more measurements of external pH of an embryo during culture show that the external pH is lower than a reference pH range for a healthy/normal embryo, indicating that the embryo is unhealthy or stressed, the embryo is graded with a lower ranking for transfer. In such cases, the methods may be used to further rank the embryos in the group based on how low the external pH is for each embryo, wherein the lower the external pH, the lower the ranking of the embryo for transfer.

Similarly, the methods described herein may also find use in ranking embryos based on their level of oxidative stress. For example, in some instances multiple embryos will be found to show elevated levels of oxidative stress markers. However, some of the embryos will be less severe than others. For example, some embryos may have elevated levels of oxidative stress markers (i.e. above reference levels for a healthy embryo), but not to the same extent as the more severely stressed embryos in the group having even higher levels of the oxidative stress markers.

In some embodiments, a combination of measurements is used to rank the embryos. For example, external pH in combination with intracellular or extracellular levels of pyruvate, lactate, or other electrolytes and/or oxidative stress markers are measured for each embryo, and used to determine the developmental potential of the embryos relative to one another. In addition, these methods may be combined with any other known method for assessing viability or developmental potential of embryos. For example, a practitioner may further measure cellular parameters or gene expression levels of one or more genes in the embryo, genotype an embryo, detect aneuploidy, quantitate blastomere fragmentation, detect blastomere asymmetry, or evaluate morphology of the embryo in culture, or any combination thereof. In some embodiments, the measurements for each of the embryos are compared directly to one another to determine the developmental potential of the embryos. In some embodiments, the measurements for each of the embryos are compared to a reference embryo to determine the developmental potentials for each embryo, and then developmental potentials for each embryo are compared to determine the developmental potential of the embryos relative to one another. In this way, a practitioner assessing, for example, multiple embryos, can choose the healthiest, least stressed, best quality embryos, i.e. those with the best developmental potential, to transfer so as to maximize the chance of success of a full-term pregnancy while minimizing risk.

For the purposes of in vitro fertilization and intracytoplasmic sperm injection, measurements of pH, pyruvate, lactate, and other electrolytes, oxidative stress markers, or other cellular parameters can occur at any stage of embryo development, from day 1 post fertilization to day 7 post fertilization or longer. An embryo is typically, though not necessarily, transferred at the blastocyst stage (after 5 days of development). At times, it is considered advantageous that the embryo be transferred to the uterus earlier in development, e.g. by day 2 or day 3, i.e., up through the 8-cell stage, to reduce embryo loss due to disadvantages of culture conditions relative to the in vitro environment, and to reduce potential adverse outcomes associated with epigenetic errors that may occur during culturing (Katari et al. (2009) Hum Mol Genet. 18(20):3769-78; Sepúlveda et al. (2009) Fertil Steril. 91(5):1765-70). Accordingly, the measurements of pH, pyruvate, lactate, and other electrolytes, oxidative stress markers, or other cellular parameters may take place within 1 day of fertilization, although longer periods of analysis, e.g., about 36 hours, about 48 hours, about 54 hours, about 60 hours, about 72 hours, about 84 hours, about 96 hours, about 120 hours, about 144 hours, about 168 hours or more, are also contemplated by the present methods.

### Reporting Assessment of an Embryo

In some embodiments, the assessment of an embryo includes generating a written report that includes an assessment of the subject embryo, e.g., “an assessment of developmental potential”, “an assessment of viability”, “an assessment of embryo health”, etc. Thus, a subject method may further include a step of generating or outputting a report providing the results of such an assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to an assessment arrived at by methods described herein. A subject report can be completely or partially electronically generated. A subject report includes at least an assessment of the developmental potential or health of the subject embryo, an assessment of oxidative stress, etc. A subject report can further include one or more of: 1) information regarding the testing facility; 2) service provider information; 3) subject data; 4) sample data; 5) a detailed assessment report section, providing information relating to how the assessment was arrived at, e.g., a) analysis of external pH, electrolytes, oxidative stress markers, and optionally other cell parameter measurements taken, b) reference values employed, if any; and 6) other features.

The report may include information about the testing facility, which information is relevant to the hospital, clinic, or laboratory in which sample gathering and/or data generation was conducted. Sample gathering can include how the sample was generated, e.g., how it was harvested from a subject, and/or how it was cultured etc. Data generation can include how pH, intracellular or extracellular levels of pyruvate, lactate or other electrolytes, and/or oxidative stress markers were analyzed. This information can include one or more details relating to, for example, the name and location of the testing facility, the identity of the lab technician who conducted the measurement and/or who entered the input data, the date and time the measurement was conducted and/or analyzed, the location where the sample and/or result data is stored, the lot number of the reagents (e.g., kit, etc.) used in assaying oxidative stress markers, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample preparation and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including medical history of subjects from which oocytes or pluripotent cells were harvested, patient age, in vitro fertilization or intracytoplasmic sperm injection cycle characteristics (e.g., fertilization rate, day 3 follicle stimulating hormone (FSH) level), and, when oocytes are harvested, zygote/embryo cohort parameters (e.g. total number of embryos). This subject data may be integrated to improve embryo assessment and/or help determine the optimal number of embryos to transfer. The report may also include administrative subject data (that is, data that are not essential to the assessment of embryo health or developmental potential) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the assessment of developmental potential and, if different from the ordering physician, the name of a staff physician who is responsible for the subject's care (e.g., primary care physician).

The report may include a sample data section, which may provide information about the biological sample analyzed in the assessment, such as the type of sample (embryo), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information relating to how the assessments/determinations were arrived at as described herein. The interpretive report can include, for example, time-lapse images of the embryo being assessed, and/or gene expression results. The assessment portion of the report can optionally also include a recommendation(s) section. For example, where the results indicate favorable developmental potential of an embryo, the recommendation can include a recommendation that a limited number of embryos be transplanted into the uterus during fertility treatment as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., an assessment of embryo health and developmental potential).

### Kits

Also provided are kits comprising a culture dish comprising sensors for measuring external pH of embryos and/or electrolytes, as described herein. In some embodiments, the culture dish is contained in a sterile package. In some embodiments, the kit includes multiple culture dishes, which can be used in culturing multiple embryos. The kit may also include one or more reagents for measuring oxidative stress markers. In addition, the kit may include culture media, incubators, microscopes, and/or other reagents or equipment for culturing or evaluating embryos.

In addition to the above components, the subject kits may further include (in certain embodiments) instructions for practicing the subject methods. In some embodiments, instructions for using the culture dish for culturing embryos and measuring external pH, electrolytes, and/or oxidative stress markers for evaluating the viability of an embryo are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-20 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A system for measuring pH of an embryo in in vitro culture, the system comprising:
a) a device comprising a reservoir for holding an embryo;
b) a pH sensor, wherein the pH sensor is located at the bottom of the reservoir; and
c) a sensor reader that displays information from the pH sensor, wherein the sensor reader is connected to the pH sensor.
2. The system of aspect 1, further comprising one or more sensors for measuring one or more electrolytes, wherein the one or more sensors are located at the bottom of the reservoir.
3. The system of aspect 2, wherein the one or more electrolytes are lactate, pyruvate, or a combination thereof.
4. The system of aspect 2 or 3, wherein the sensor reader further displays information about the levels of the electrolytes.
5. The system of any one of aspects 1 to 4, further comprising an oxygen sensor.
6. The system of any one of aspects 1 to 5, further comprising a temperature sensor.
7. The system of aspect 5 or 6, wherein the sensor reader further displays information about the level of oxygen and/or temperature.
8. The system of any one of aspects 1 to 7, further comprising one or more of an incubator, microscope, and culture media.
9. The system of any one of aspects 1 to 8, wherein the pH sensor comprises an electrode or a pH-sensitive dye or other pH indicator.
10. A method of measuring pH during in vitro culture of an embryo and implanting the embryo into a female recipient if the embryo is not determined to have poor developmental potential based on the pH, the method comprising:
a) culturing an embryo in vitro in a culture media under conditions suitable for embryo development in the reservoir of the system of any one of aspects 1 to 9;
b) measuring pH of the culture media in the reservoir during development of the embryo at one or more time points using the sensor reader of the system of any one of aspects 1 to 9, wherein a lower pH of the culture media compared to a reference value for a healthy embryo indicates that the embryo has poor developmental potential; and
c) implanting the embryo into a female recipient if the embryo is not determined to have poor developmental potential based on the pH.
11. The method of aspect 10, further comprising measuring levels of one or more markers of oxidative stress in a cell of the embryo, wherein lower pH of the culture media in combination with higher levels of the one or more markers of oxidative stress compared to reference value ranges for a healthy embryo indicate that the embryo has poor developmental potential.
12. The method of aspect 11, wherein the oxidative stress markers are protein oxidative stress markers, DNA oxidative stress markers, RNA oxidative stress markers, and lipid oxidative stress markers.
13. The method of aspect 12, wherein the oxidative stress markers are selected from the group consisting of proteins with carbonyl oxidation, 8-hydroxyguanosine (8-OHG), 8-hydroxydeoxyguanosine (8-OHdG), 8-nitroguanine, abasic (AP) sites, ethenoadenosine, ethenocytidine, ethenoguanine, ethenodeoxyadenosine, ethenodeoxycytidine, ethenodeoxyguanosine, 8-iso-prostaglandin F2alpha (8-isoprostane), 4-hydroxynonenal (HNE), and malondialdehyde (MDA).
14. The method of any one of aspects 11 to 13, further comprising measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with lower pH of the culture media and higher levels of the one or more markers of oxidative stress compared to reference value ranges for a healthy embryo indicate that the embryo has poor developmental potential.
15. The method of any one of aspects 10 to 14, wherein the embryo is human.
16. A method for evaluating the developmental potential of an embryo, the method comprising measuring internal pH of the embryo during development, wherein a higher internal pH of the embryo compared to a reference value for a healthy embryo indicates that the embryo has poor developmental potential.
17. The method of aspect 16, further comprising measuring one or more markers of oxidative stress in a cell of the embryo, wherein higher internal pH of the embryo in combination with higher levels of the one or more markers of oxidative stress compared to reference value ranges for a healthy embryo indicate that the embryo has poor developmental potential.
18. The method of aspect 17, further comprising measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with higher internal pH in the embryo and higher levels of the one or more markers of oxidative stress compared to reference value ranges for a healthy embryo indicate that the embryo has poor developmental potential.
19. The method of any one of aspects 10 to 18, further comprising measuring one or more cellular parameters, measuring gene expression levels of one or more genes in the embryo, genotyping the embryo, detecting aneuploidy, quantitating blastomere fragmentation, detecting blastomere asymmetry, or evaluating morphology of the embryo in culture, or any combination thereof.
20. A kit comprising the system of any one of aspects 1 to 9.
21. The kit of aspect 20, further comprising instructions for using the culture dish for measuring external pH while culturing an embryo to evaluate the viability of an embryo.
22. The kit of aspect 20 or 21 further comprising at least one reagent for measuring levels of an oxidative stress marker.
23. The kit of any one of aspects 20 to 22, further comprising at least one reagent for measuring levels of lactate or pyruvate.
24. The kit of any one of aspects 20 to 23, further comprising media for culturing an embryo.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

A Device for Measuring pH Noninvasively during Culture of an Embryo Generated by IVF for Assessment of Viability

### Introduction

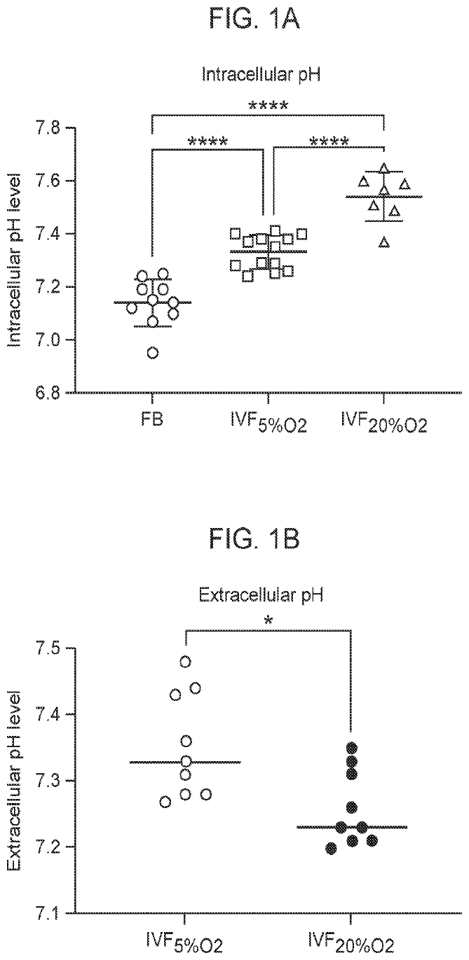

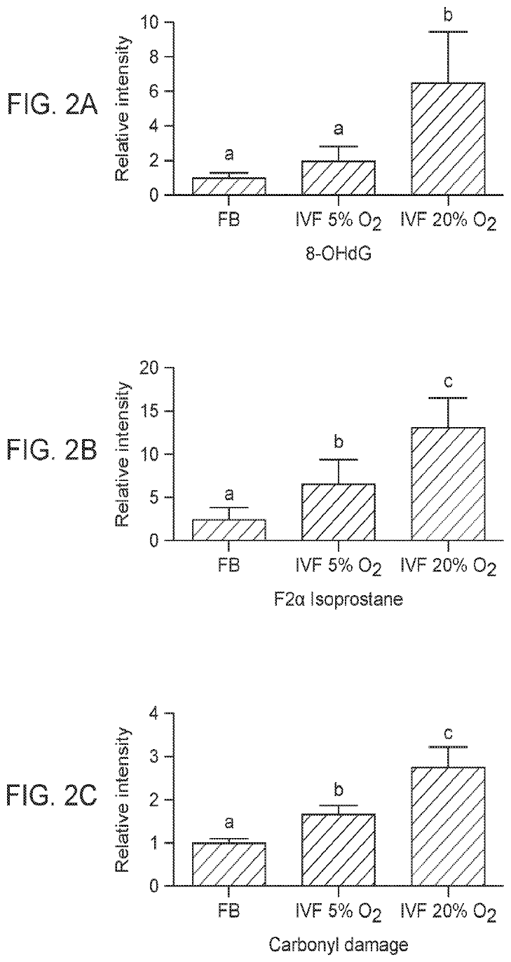

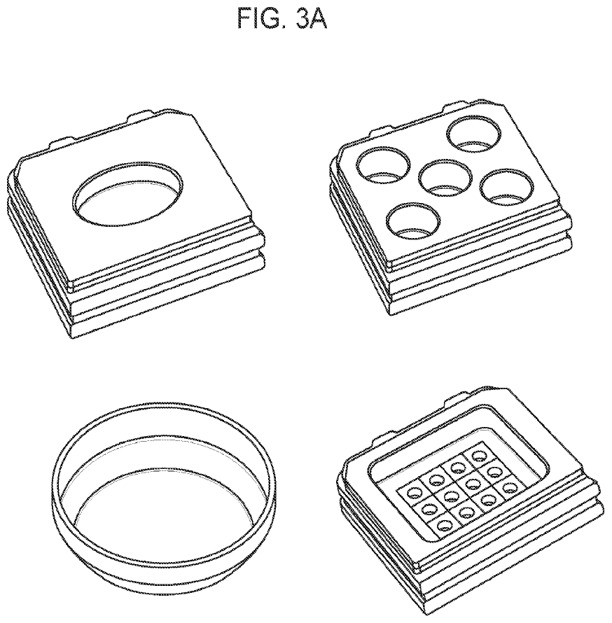

Our more recent studies indicate that mitochondrial and oxidative stress are present in preimplantation mouse embryos generated by IVF. In particular, mouse embryos generated in vitro produce more reactive oxygen species (ROS) and display altered mitochondria number with decreased membrane potential and decreased levels of mitochondrial DNA [mtDNA (Belli, et al. 2019)]. We have discovered that if the culture conditions are stressful (i.e. farther away from the in vivo physiologic conditions): 1) the higher the internal pH (pHi) of preimplantation embryos and the lower the pH in the culture medium (pHe) (FIG. 1). This is similar to what happens to cancer cells: cancer cells, which display profoundly altered metabolism, have higher pHi and lower pHe (Webb, et al. 2011). Similarly, with increasing suboptimal culture conditions, we found higher levels of protein oxidative stress (measured by carbonyl oxidation of protein), lipid oxidative stress (measured by F2alpha isoprostane), DNA oxidative stress (measured by levels of 8 hydroxy-deoxy-guanosine) (FIG. 2). These observations suggest that embryos can be graded with respect to their health by measuring their metabolic stress. This grading system will enable the selection of embryos with a low metabolic stress pattern (more similar to in vivo embryos) to transfer during human IVF.

IVF-generated embryos with pHi similar to in vivo generated embryos or with lower oxidative stress are likely to be the healthiest embryos to transfer. Therefore, the findings that mouse embryos with higher stress have higher pHi and secrete more H⁺ ions resulting in lower pHe, (FIG. 1) are relevant because: 1) we are able to measure pHe in culture drops where individual human embryos are cultured; 2) we can select the most viable embryo based on pHe. Similarly, the findings that embryos with more stress show increased oxidative damage indicate that measuring oxidative damage in selected cells of embryos is also useful in identifying viable embryos for transfer.

We are developing a device to measure reliably and non-invasively pHe in drops where individual embryos are cultured. Mouse and human embryos are routinely cultured in 20-30 μl drops for 3 to 5 days and then transferred to the uterus. The culture media where the embryo is grown is normally discarded. Instead, we measure pH in the drop (i.e. pHe) where the embryo was cultured; this will provide key information on the metabolism and health of the embryo. While pH-meter and pH probes are widely available, they are cumbersome, fragile and therefore not appropriate to use in the setting of an IVF clinic. Instead, we use a novel miniaturized pH sensor device to measure pH in situ (FIG. 3B). This device is uniquely tailored for the particular conditions used in human IVF.

### Example 2

### Measuring Oxidative Stress in Cells of an Embryo Generated by IVF for Assessment of Viability

We also found that with increasing suboptimal culture conditions, embryo display higher levels of protein oxidative stress (measured by carbonyl oxidation of protein), lipid oxidative stress (measured by F2alpha isoprostane), and DNA oxidative stress (measured by levels of 8 hydroxy-deoxy-guanosine) (FIG. 2). These observations suggest that embryos can be graded with respect to their health by measuring their metabolic stress. This grading system enables the selection of embryos with a low metabolic stress pattern (more similar to in vivo embryos) to transfer during human IVF.

Currently approximately 20% of human embryos undergo a procedure called preimplantation genetic testing (PGT), where 3-5 cells are removed to understand if the embryo has a normal set of chromosomes (Dandouh, et al. 2015). Once the DNA is obtained, the rest of the biopsied cells are discarded. Instead of discarding this precious material, oxidative damage can be measured in these cells. Embryos with lower oxidative stress are considered the healthiest to transfer.

### Example 3

Measuring Electrolytes during Culture of an Embryo Generated by IVF for Assessment of Viability

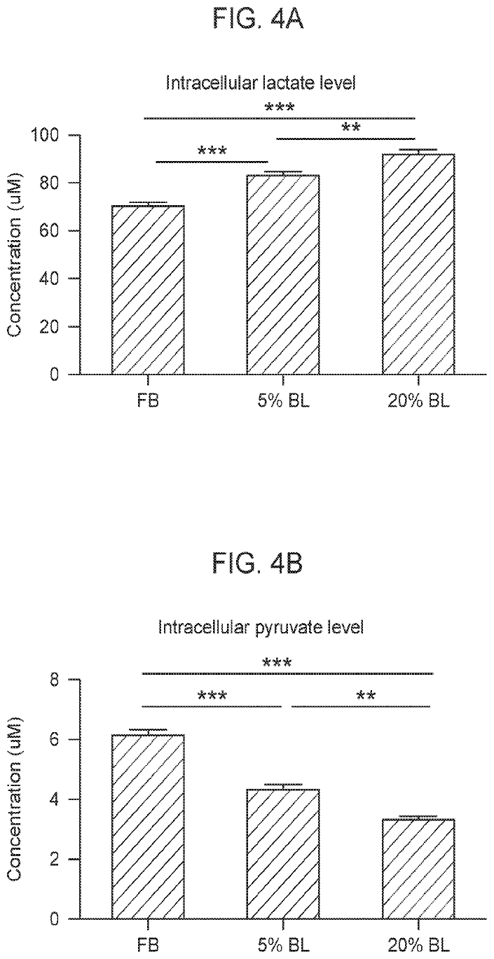

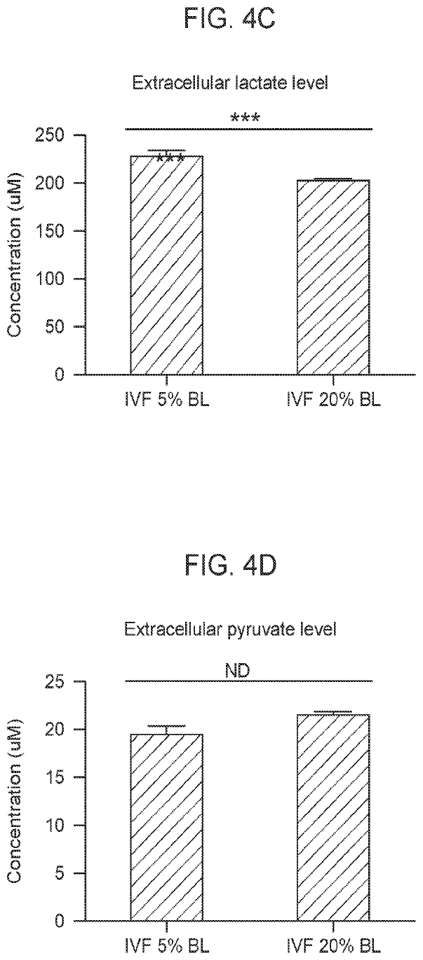

We also measured lactate and pyruvate levels in embryos and culture media of embryos. Intracellular lactate levels increased with increasing suboptimal culture conditions (FIG. 4A). This signifies that the healthiest embryo can be identified based on intracellular lactate levels. A healthy IVF-generated embryo has a lactate level closer to an in vivo (flashed blastocyst (FB)=control) embryo. Extracellular lactate levels decreased with increasing suboptimal culture conditions (FIG. 4C). This means that extracellular level of lactate can also be used to identify the healthiest embryo.

Intracellular pyruvate levels decreased with increasing suboptimal culture conditions (FIG. 4B). This signifies that the healthiest embryo can be identified based on intracellular pyruvate levels. A healthy IVF-generated embryo has a pyruvate level closer to an in vivo (flashed blastocyst (FB)=control) embryo. Extracellular pyruvate levels did not change significantly (ND=not different statistically) with increasing suboptimal culture conditions (FIG. 4D). This means that extracellular levels of pyruvate may not be useful in identifying the healthiest embryo.

Currently approximately 20% of human embryos undergo a procedure called preimplantation genetic testing (PGT), where 3-5 cells are removed to understand if the embryo has a normal set of chromosomes (Dandouh, et al. 2015). Once the DNA is obtained, the rest of the biopsied cells are discarded. Instead of discarding this precious material, oxidative damage (i.e., as indicated by levels of oxidative stress markers) in these cells can be measured, as we have done in FIG. 2, as well as intracellular pyruvate and lactate levels. In addition, we are investigating the molecular mechanisms of control of pHi and are measuring metabolites of glycolysis (in addition to lactate and pyruvate) both intracellularly and in culture media.

### LITERATURE CITATIONS

Adamson, G D, F Zegers-hochschild, S Dyer, G Chambers, O Ishihara, R Mansour, M Banker, M Banker, and J de Mouzoun 2018 International Committee for Monitoring Assisted Reproductive Technologies (ICMART) World Report on ART, 2014 and The International Glossary on Infertility and Fertility Care, 2017. ESHRE.
Bateson, P, D Barker, T Clutton-Brock, D Deb, B D'Udine, R A Foley, P Gluckman, K Godfrey, T Kirkwood, M M Lahr, J McNamara, N B Metcalfe, P Monaghan, H G Spencer, and S E Sultan 2004 Developmental plasticity and human health. Nature 430 419-421.
Belli, M, L Zhang, X Liu, A Donjacour, E Ruggeri, M G Palmerini, S A Nottola, G Macchiarelli, and P Rinaudo 2019 Oxygen concentration alters mitochondrial structure and function in in vitro fertilized preimplantation mouse embryos. Hum Reprod 34 601-611.
Bloise, E, W Lin, X Liu, R Simbulan, K S Kolahi, F Petraglia, E Maltepe, A Donjacour, and P Rinaudo 2012 Impaired placental nutrient transport in mice generated by in vitro fertilization. Endocrinology 153 3457-3467.
Ceelen, M, M M van Weissenbruch, J P Vermeiden, F E van Leeuwen, and H A Delemarre-van de Waal 2008 Cardiometabolic differences in children born after in vitro fertilization: follow-up study. J Clin Endocrinol Metab 93 1682-1688.
Dandouh, E M, J Balayla, and J A Garcia-Velasco 2015 Impact of blastocyst biopsy and comprehensive chromosome screening technology on preimplantation genetic screening: a systematic review of randomized controlled trials. Reprod Biomed Online 30 281-289.
Delle Piane, L, W Lin, X Liu, A Donjacour, P Minasi, A Revelli, E Maltepe, and P F Rinaudo 2010 Effect of the method of conception and embryo transfer procedure on mid-gestation placenta and fetal development in an IVF mouse model. Hum Reprod 25 2039-2046.
Donjacour, A, X Liu, W Lin, R Simbulan, and P F Rinaudo 2014 In vitro fertilization affects growth and glucose metabolism in a sex-specific manner in an outbred mouse model. Biol Reprod 90 80.
Feuer, S, X Liu, A Donjacour, R Simbulan, E Maltepe, and P Rinaudo 2016 Common and specific transcriptional signatures in mouse embryos and adult tissues induced by in vitro procedures. Reproduction 153 107-122.
Feuer, S K, X Liu, A Donjacour, W Lin, R K Simbulan, G Giritharan, L D Piane, K Kolahi, K Ameri, E Maltepe, and P F Rinaudo 2014 Use of a mouse in vitro fertilization model to understand the developmental origins of health and disease hypothesis. Endocrinology 155 1956-1969.
Meister, T A, S F Rimoldi, R Soria, R von Arx, F H Messerli, C Sartori, U Scherrer, and E Rexhaj 2018 Association of Assisted Reproductive Technologies With Arterial Hypertension During Adolescence. J Am Coll Cardiol 72 1267-1274.
Rimoldi, S F, C Sartori, E Rexhaj, D M Bailey, S F Marchi, J McEneny, R V Arx, D Cerny, H Duplain, M Germond, Y Allemann, and U Scherrer 2014 Antioxidants improve vascular function in children conceived by assisted reproductive technologies: A randomized double-blind placebo-controlled trial. Eur J Prev Cardiol.
Rinaudo, P, and R M Schultz 2004 Effects of embryo culture on global pattern of gene expression in preimplantation mouse embryos. Reproduction 128 301-311.
Webb, B A, M Chimenti, M P Jacobson, and D L Barber 2011 Dysregulated pH: a perfect storm for cancer progression. Nat Rev Cancer 11 671-677.

1. A system for measuring pH of an embryo in in vitro culture, the system comprising:
a) a device comprising a reservoir for holding an embryo;
b) a pH sensor, wherein the pH sensor is located at the bottom of the reservoir; and
c) a sensor reader that displays information from the pH sensor, wherein the sensor reader is connected to the pH sensor.

2. The system of claim 1, further comprising one or more of:
one or more sensors for measuring one or more electrolytes, wherein the one or more sensors for measuring one or more electrolytes are located at the bottom of the reservoir;
an oxygen sensor; and
a temperature sensor.

3. The system of claim 2, wherein the one or more electrolytes are lactate, pyruvate, or a combination thereof.

4. The system of claim 2, wherein the sensor reader further displays information about the levels of the electrolytes, oxygen, and/or temperature.

5-7. (canceled)

8. The system of claim 1, further comprising one or more of an incubator, microscope, and culture media.

9. The system of claim 1, wherein the pH sensor comprises an electrode or a pH-sensitive dye or other pH indicator.

10. A method of measuring pH during in vitro culture of an embryo and implanting the embryo into a female recipient if the embryo is not determined to have poor developmental potential based on the pH, the method comprising:
a) culturing an embryo in vitro in a culture media under conditions suitable for embryo development in the reservoir of the system of claim 1;
b) measuring pH of the culture media in the reservoir during development of the embryo at one or more time points using the sensor reader of the system of claim 1, wherein a lower pH of the culture media compared to a reference value for a healthy embryo indicates that the embryo has poor developmental potential; and
c) implanting the embryo into a female recipient if the embryo is not determined to have poor developmental potential based on the pH.

11. The method of claim 10, further comprising measuring levels of one or more markers of oxidative stress in a cell of the embryo, wherein lower pH of the culture media in combination with higher levels of the one or more markers of oxidative stress compared to reference value ranges for a healthy embryo indicate that the embryo has poor developmental potential.

12. The method of claim 11, wherein the oxidative stress markers are protein oxidative stress markers, DNA oxidative stress markers, RNA oxidative stress markers, and lipid oxidative stress markers.

13. The method of claim 12, wherein the oxidative stress markers are selected from the group consisting of proteins with carbonyl oxidation, 8-hydroxyguanosine (8-OHG), 8-hydroxydeoxyguanosine (8-OHdG), 8-nitroguanine, abasic (AP) sites, ethenoadenosine, ethenocytidine, ethenoguanine, ethenodeoxyadenosine, ethenodeoxycytidine, ethenodeoxyguanosine, 8-iso-prostaglandin F2alpha (8-isoprostane), 4-hydroxynonenal (HNE), and malondialdehyde (MDA).

14. The method of claim 11, further comprising measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with lower pH of the culture media and higher levels of the one or more markers of oxidative stress compared to reference value ranges for a healthy embryo indicate that the embryo has poor developmental potential.

15. The method of claim 10, wherein the embryo is human.

16. A method for evaluating the developmental potential of an embryo, the method comprising measuring internal pH of the embryo during development, wherein a higher internal pH of the embryo compared to a reference value for a healthy embryo indicates that the embryo has poor developmental potential.

17. The method of claim 16, further comprising measuring one or more markers of oxidative stress in a cell of the embryo, wherein higher internal pH of the embryo in combination with higher levels of the one or more markers of oxidative stress compared to reference value ranges for a healthy embryo indicate that the embryo has poor developmental potential.

18. The method of claim 17, further comprising measuring intracellular or extracellular levels of lactate, intracellular levels of pyruvate, or a combination thereof, wherein increased intracellular levels of lactate, decreased extracellular levels of lactate, or decreased intracellular levels of pyruvate in combination with higher internal pH in the embryo and higher levels of the one or more markers of oxidative stress compared to reference value ranges for a healthy embryo indicate that the embryo has poor developmental potential.

19. The method of claim 16, further comprising measuring one or more cellular parameters, measuring gene expression levels of one or more genes in the embryo, genotyping the embryo, detecting aneuploidy, quantitating blastomere fragmentation, detecting blastomere asymmetry, or evaluating morphology of the embryo in culture, or any combination thereof.

20. A kit comprising the system of claim 1.

21. The kit of claim 20, further comprising instructions for using the culture dish for measuring external pH while culturing an embryo to evaluate the viability of an embryo.

22. The kit of claim 20 further comprising at least one reagent for measuring levels of an oxidative stress marker and/or at least one reagent for measuring levels of lactate or pyruvate.

23. (canceled)

24. The kit of claim 20, further comprising media for culturing an embryo.
